(19) United States
(12) Patent Application Publication
(10) Pub. No.: US 2015/0045422 A1
(43) Pub. Date: Feb. 12, 2015

(54) Influenza treatment using targeted transient ribosomal inhibition

(71) Applicant: Mark John Zamoyski, San Jose, CA (US)

(72) Inventor: Mark John Zamoyski, San Jose, CA (US)

(21) Appl. No.: 13/385,052

(22) Filed: Jan. 31, 2012

Related U.S. Application Data
(62) Division of application No. 12/321,717, filed on Jan. 24, 2009, now abandoned.
Application No. 12/321,717 is a continuation-in-part of application No. 11/159,611, filed on Jun. 22, 2005, now abandoned.

(51) Int. Cl. A61K 31/35; A61P 31/16

(52) U.S. Cl. 514/453

(57) ABSTRACT

Novel methods are presented for treating Influenza by using Targeted Transient Ribosomal Inhibition (TTRI), with TTRI drugs administered by inhalation, to silence expression of every protein underlying the etiology and pathology of the influenza infection, and without adversely effecting normal cells in the lungs. The therapeutic effect is dual Antiviral and Anti-Inflammatory activity which is localized to the lungs. Pulmonary TTRI for influenza silences expression of all 10 influenza viral proteins and de novo synthesis of cytokines and leukotrienes produced in the lungs. By targeting ribosomes, TTRI is insensitive to viral mutations or human alterations to the virus. TTRI provides an off-the-shelf solution for preventing mortality from any strain of influenza.

### CROSS REFERENCE TO RELATED APPLICATIONS

The current application is a Divisional application of Ser. No. 12/321,717 filed on Jan. 24, 2009 which was a Continuation In Part of application Ser. No. 11/159, 611 filed on Jun. 22, 2005. The parent application (Ser. No. 12/321,717) presented Targeted Transient Ribosomal Inhibition as a novel method of treating of Herpes Lesions, Influenza, and Acne. Examiner required limitation of the claims in application to a single indication, which required filing Divisional applications for the other two indications. This Divisional application is for use of Targeted Transient Ribosomal Inhibition for treatment of Influenza.

Current invention teaches administration of trichothecenes by inhalation as a dual action antiviral and anti-inflammatory treatment for influenza.

### STATEMENT REGARDING FEDERALLY SPONSORED RESEARCH

Not Applicable.

### INCORPORATION-BY-REFERENCE OF MATERIAL SUBMITTED ON A CD

Not Applicable

### BACKGROUND OF THE INVENTION

### Field of the Invention

Trichothecenes are small molecules (˜300-600 MW) made of only carbon, hydrogen, and oxygen. They possess a central sesquiterpene epoxide structure and are categorized as Type A, B, C, or D. They freely cross plasma membranes and bind with high affinity to the ribosomal peptidyl transferase site, preventing the translation of RNA into proteins (Victor I. Shifrin and Paul Anderson of Brigham and Women's Hospital, The Journal of Biological Chemistry, Vol. 274, No. 20, Issue of May 14, pp. 13985-13992, 1999). As used in this application, and its related claims, the terms inhibition of RNA (RNAi), or proteins synthesis restriction (PSR), or protein synthesis inhibition (PSI) are used interchangeably to described this activity. In general, Type D trichothecenes are the most potent, followed by Type A, B, and C respectively. The trichothecene used in the representative examples of present invention is diacetoxyscirpenol (DAS), a type A trichothecene.

As will be disclosed, the therapeutic affect of this type of RNAi/PSR is Antiproliferative, Antiviral, and Anti-Inflammatory activity. As will also be disclosed, this type of RNAi/PSR can be sustained continuously for at least three days without any reduction in cell viability.

Prior art's use of trichothecene has been limited to cell cycle active cytotoxic doses for treating cancer and dates back to the early 1980s. Anguidine (aka diacetoxyscirpenol or DAS), a simple trichothecene, was administered in cytotoxic doses, however its use was abandoned after Phase II results showed overall tumor response rate was low and there was considerable hematologic toxicity. U.S. Pat. Nos. 4,744,981 and 4,906,452 embody the direction prior art took to solve the systemic toxicity problem caused by trichothecene's lack of specificity in cellular internalization and blood insolubility; they proposed using conjugates of trichothecene with monoclonal or polyclonal antibodies to selectively deliver the toxin to tumors and proposed glycosylation of trichothecenes to increase blood solubility. Applicant took an exactly opposite approach to prior art and demonstrated how certain trichothecenes could be used unconjugated and unglycosylated to treat tumors by reversing the direction of administration from tissue to blood (interstitial perfusion), dispersing the trichothecenes between the intercellular spaces and then using the gap junction transport system to cleanly localize the trichothecene in the tumor, for which applicant was granted U.S. Pat. No. 6,342,520.

Applicant demonstrated that because of the molecules' extremely small size, lipophilic nature, and blood insolubility, the molecules could be used as a topical treatment for psoriasis, for which applicant was granted U.S. Pat. No. 6,346,251. Applicant also demonstrated that the molecules could be used for epidermal chemexfoliation, which provided utility for treating numerous medical conditions, for which applicant was granted U.S. Pat. No. 6,355,251. Applicant also demonstrated how these molecules could also be used by inhalation, as a Chemodebridement for COPD for which applicant was granted U.S. Pat. No. 7,015,244 and as an inhibitor of inflammation in the respiratory tract for which applicant was granted U.S. Pat. No. 7,012,091.

In the present invention, applicant teaches dual action anti-infective, anti-inflammatory treatment methods for dermal disease conditions such as acne and herpes lesions and pulmonary conditions including pernicious strains of influenza (Avian flu, Spanish Flu) and chronic viral infections associated with COPD.

### BRIEF SUMMARY OF THE INVENTION

Present invention will provide novel, dual action, anti-infective and anti-inflammatory treatment methods for dermal and pulmonary conditions. Representative examples presented include dermal conditions such as Herpes lesions and Acne and pulmonary conditions such as influenza (H5N1).

### BRIEF DESCRIPTION OF DRAWING FIGURES

FIG. 1a shows the antiproliferative cytostatic/cytotoxic profile for DAS in cancer cell lines and FIG. 1b shows the antiproliferative cytostatic/cytotoxic profile for DAS in non cancer cell lines.

FIG. 2 shows the antiviral activity profile for DAS and T-2 in human epidermoid cells infected with HSV2

FIG. 3 shows the anti-inflammatory activity of PSR in mouse models

FIG. 4 shows the dosimetry of inhaled particles

FIG. 5 shows the H5N1 disease progression timeline versus a primary immune response timeline

FIG. 6 shows the safety of topical and inhaled doses of DAS relative the systemic NOEL dose of DAS for the representative examples provided in the present application.

### DETAILED DESCRIPTION OF THE INVENTION

The treatments disclosed below involve topical or inhalable administration of biologically active trichothecenes to inhibit both an underlying pathogen and the immune mediated inflammation in response to the underlying pathogen. Materials and methods for achieving this are described below.

### Trichothecenes Defined:

Fungi of the genera Fusarium, Myrotecium, Trichoderma, Stachybotrys and others produce Trichothecenes. Over 150 trichothecenes have been identified and approximately 50 synthetic variants have been made. There are two broad classes: those that have only a central sesquiterpenoid structure and those that have an additional macrocyclic ring (simple and macrocyclic trichothecenes, respectively). As used in this application, and its related claims, “therapeutics”, “biologically active agent”, or “trichothecene” are defined as either simple or macrocyclic trichothecenes, or a any fragment or subunit thereof that still retains the biological function of inhibiting translation of RNA into proteins, and include molecules of the following general chemical formulas:

Simple trichothecenes are categorized into three groups with the following chemical formulas:

### Group A:

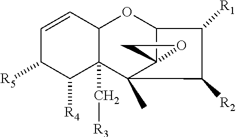

### Wherein R₁ is H, OH, or

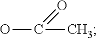

### R₂ is H, OH, or

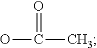

### R₃ is H, OH, or

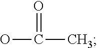

### R₄ is H or OH; and

### R₅ is H, OH,

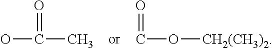

### Group B:

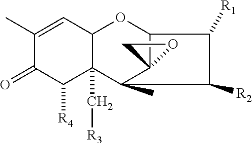

### Wherein R₁ is H, OH, or

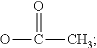

### R₂ is H, OH,

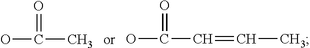

### R₃ is H, OH, or

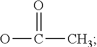

### R₄ is H, OH, or

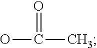

### Group C:

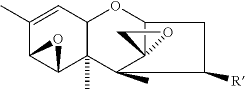

### Wherein R′ is OH or

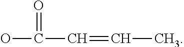

Macrocyclic Trichothecenes can be described by the following general chemical formulas:

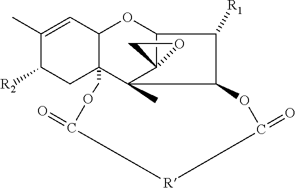

Wherein R₁ is OH, or

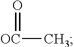

R₂ is H, OH,

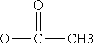

### or OCOCH₂CH(CH₃)₂; and

R′ is any molecule composed predominantly, or in its entirety, of combinations of C, H, and O:

Some representative examples of R′ include:
Satratoxin H:

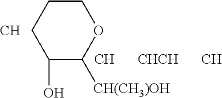

Satratoxin G:

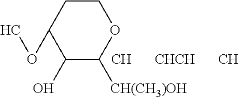

or molecules of the following general formula:

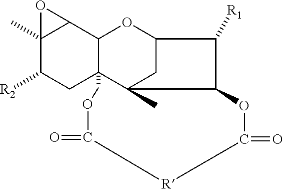

Wherein R₁ is H, OH, or

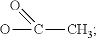

R₂ is H, OH,

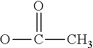

### or OCOCH₂CH(CH₃)₂; and

R′ is any molecule composed predominantly, or in its entirety, of combinations of C, H, and O.

A more comprehensive listing of trichothecenes is included in U.S. Pat. No. 4,906,452, incorporated herein by reference.

Trichothecenes are invisible to the immune system since they neither contain nor produce amino acids. Since trichothecene molecules contain only carbon, hydrogen, and oxygen they are not subject to proteolytic degradation. U.S. Pat. No. 4,906,452 (column 11 first paragraph) further discloses that some studies of the rates at which certain trichothecenes are converted into biologically inactive molecules (apotrichothecenes) found that macrocyclic trichothecenes are inactivated quite slowly and only by intracellular acid catalysis as might occur in lyzosomes.

Trichothecenes are extremely stable to heat and ultraviolet light inactivation. Heating to 500° F. for 30 minutes is required for inactivation. Brief exposure to NaOH destroys toxic activity. These substances are relatively insoluble in water but are highly soluble in ethanol, methanol, and propylene glycol.

Trichothecenes also have a second, weaker mechanism of action (MOA) of sulphydryl binding, which does manifest until much higher concentrations are used. Sulphydryl groups are primarily involved at a sub cellular level with transport of structural proteins and concentration gradient maintenance. Sulphydryl restriction (SHR) activity is associated with edema, neural impairment, and at extremely high doses cellular necrosis. Type A trichothecene such as DAS and T-2 have potent PSR activity and very low sulphydryl restriction (SHR) activity. The doses used in representative examples are several multiples below any observable SHR activity (discussed in detail later) and as such the dose computations contained herein are limited to the relevant RNAi/PSR MOA.

### MATERIALS AND METHODS OF PRESENT INVENTION

Trichothecenes can be obtained by various methods as described by applicant in U.S. Pat. No. 7,015,244 col. 7 last paragraph to col. 8 second paragraph, incorporated herein by reference.

The preferred trichothecene used in representative examples of instant invention is DAS. The method described by Richardson & Hamilton (Kurt E. Richardson and Pat B. Hamilton, “Preparation of 4,15-Diacetoxyscirpenol from Cultures of Fusarium sambucinum NRRL 13495”, Applied and Environmental Microbiology, February 1987, p 460-462) results in ˜0.5 g of DAS per liter of culture after 9 days. From the abstract: “Filtrates of Fusarium sambucinum NRRL 13495 grown in a stagnant culture for 9 days contained up to 458±60 (mean±standard error; n=3) mg of 4,15-diacetoxyscirpenol per liter depending on culture conditions. Extraction with ethyl acetate, chromatography on a column of silica gel, and crystallization from mixtures of ethyl acetate and hexane provided pure material in 96% yield.” In the late 1990's, more specialized columns for cleanup of DAS from extract became commercially available (MycoSep 225 from Romer Labs).

Although 0.5 grams of DAS per liter may not sound like much, it represent more than 50,000 typical Herpes lesion or Acne lesion doses.

DAS and T-2 are just a couple of examples of Type A trichothecenes that could be used. They have the central sesquiterpenoid structure, with only one side chain difference:

TABLE 1
DAS and T-2 Structures

| Trichothecene | MW | CAS No. | R1 | R2 | R3 | R4 | R5 |
|---|---|---|---|---|---|---|---|
| DAS | 366 | 2270-40-8 | OH | OAc | OAc | H | H |
| T-2 | 466 | 21259-20-1 | OH | OAc | OAc | H | OCOCH₂CH(CH₃)₂ |

The potency of both DAS and T-2 in human cell lines is similar. The IC50 is the concentration (in ng/ml) required to inhibit 50% of protein synthesis and is shown below:

TABLE 2
IC50 in Human Cell Lines

| | HEp2 | HL | HeLa | HEK |
|---|---|---|---|---|
| | (epidermoid) | (lymphocyte) | (uterine) | (kidney) |
| DAS | | 1 | 10 | 10 |
| T-2 | 1 | 3 | 10 | 20 |

DAS has an intracellular inactivation time of around half a day, requiring at least twice daily administration for continued efficacy. T-2 has a longer intracellular inactivation time.

However, any suitable trichothecene could be substituted for DAS or T-2. The two sets of data required for computing dosing and administration schedules are 1) the dose dependent protein synthesis inhibition profile and 2) intracellular inactivation time. Although these two attributes are disclosed for DAS above, they can readily be determined for any trichothecene, or any other RNAi, PSR, or PSI compound. In general, the procedures of W. L. Thompson and R. W. Wannemacher, Jr. are used (W. L. Thompson and R. W. Wannemacher, Jr., U.S. Army Medical Research Institute of Infectious Diseases, “Detection and Quantification of T-2 Mycotoxin with a Simplified Protein Synthesis Inhibition Assay”, Applied and Environmental Microbiology, December 1984, P. 1176-1180). As a representative example, monolayers of cells are exposed to the various concentrations of trichothecene for 15 min. The medium is removed and 2 μCi of tritium labeled leucine in medium is added for 30 min. The isotope is then removed, cells harvested, and the tritium leucine incorporation into proteins determined by scintillation counter. Results are plotted as a percentage of the protein synthesis rate in the untreated cells to yield a dose dependent protein synthesis inhibition profile. The intracellular inactivation profile is constructed by measuring the percentage of protein synthesis inhibition at several points in time after removal of the drug. Resumption of protein synthesis is effectively used as the indicator of intracellular inactivation time.

TABLE 3 below provides a frame of reference of the relative potency of the various types of trichothecenes:

TABLE 3
PSR Potency in Human Cells (IC50 in ng/ml)

| | HeLa | HEK | HL | HEp2 | HSV2 |
|---|---|---|---|---|---|
| Type A | | | | | |
| T-2 | 10 | 20 | 3 | 1 | 1.6 |
| H-T2 | 10 | 100 | 10 | | |
| DAS | 10 | 10 | 1 | | 2.3 |
| NEOS | 100 | 60 | 50 | | 52.0 |
| Type B | | | | | |
| DON | 1000 | 3000 | 500 | 250 | 94 |
| NIV | 300 | 1000 | 300 | 225 | 50 |
| FusX | 100 | 1000 | 300 | | 26 |
| Type C | | | | | |
| Crotocin | 500 | 600 | 2000 | 250 | |
| Type D | | | | | |
| Satratoxin G | | | | 2 | 1.5 |
| Satratoxin H | | | | | |
| Roridin A | 3 | 3 | 3 | 2 | 1.4 |
| Verrucarin A | 5 | 2 | 3 | 1 | |
| Baccharinoid B-4 | | | | | 41.0 |
| Baccharinoid B-5 | | | | | 9.0 |

As previously disclosed, in general, Type D trichothecenes are the most potent, however their intracellular inactivation times are extremely long, with some demonstrating PSR activity for around a week. DAS was selected for use in the preferred embodiment in part because of its much shorter intracellular inactivation time (i.e. ˜one half day) and in part because the extensive human safety data available from human clinical trials of DAS against cancer in the 1970's and 1980's. As will be discussed later, sustained PSR is a key determinant in cytostatic versus cytotoxic effect, and hence intracellular inactivation time is a key factor in selecting the right trichothecene for a given application. Long inactivation times are better for indications where cytotoxic effect is desired. Short inactivation times are better for indications were cytotoxic effect is not desired.

The therapeutic effect of RNAi/PSR is Antiproliferative, Antiviral, and Anti-inflammatory activity.

### Antiproliferative Activity:

The antiproliferative activity of DAS was perhaps best characterized by Dosik et. al. (Dosik et. al. University of Texas, Md. Anderson Hospital, “Lethal and Cytokinetic Effects of Anguidine on a Human Colon Cancer Cell Line”, Cancer Research 38, 3304-3309, October 1978). The study evaluated DAS's activity on a human adenocarcinoma cell line (LoVo). The major findings of the study were:

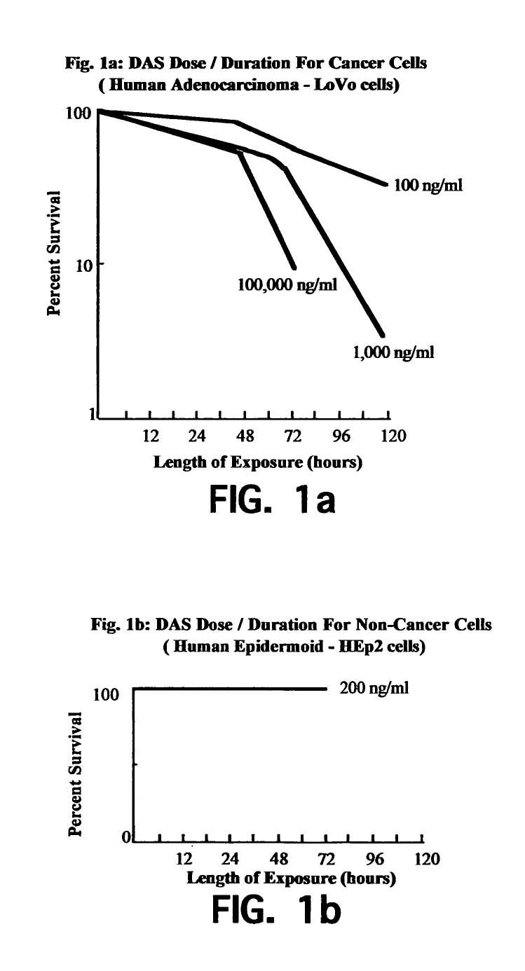

1) DAS is a G1 and G2 Phase Cytostatic. “An immediate reduction in the cumulative labeling index for cells continuously exposed to tritiated thymidine and anguidine and a rapid decrease in the cumulative mitotic index for cells continually exposed to Colcemid and anguidine indicated a block at the G1 into S and G2 into mitosis transitions.” “The kinetic data reflect an almost frozen state of the cell cycle”.
2) The Action of DAS is Reversible. “We conclude that early anguidine-induced cell cycle perturbation is reversible.”
3) Prolonged Exposure to DAS is Required for Cytotoxic Effect. FIG. 1 (excerpted from Dosik et. al., pg. 3306, Chart 2) shows duration of exposure required for cytotoxic effect.

The antiproliferative activity of DAS (anguidine, NSC 141537) was further demonstrated against transplantable mouse tumors P-388 leukemia, L-1210 leukemia, and Lewis Lung carcinoma. DAS was administered once daily for 9 consecutive days. The median survival data is summarized below by cancer type (per Bristol Myers, U.S. Pat. No. 4,244,874, Col. 10-12, Table I, II, and VI).

TABLE 4
Mouse Models - Median Survival by Cancer Type
Median Survival Time (in days)

| DAS Dose | P-388 | L-1210 | Lung |
|---|---|---|---|
| (mg/kg) | Leukemia | Leukemia | Carcinoma |
| 1.6 | 15.5 | 11.0 | 21.0 |
| 0.8 | 14.5 | 11.0 | 21.0 |
| 0.4 | 13.0 | 10.0 | 23.0 |
| 0.2 | 12.0 | 9.0 | |
| Control (saline) | 9.0 | 7.0 | 17.0 |

DAS was then tested in several Phase II human clinical trials with less than spectacular results. The results of the two largest Phase II trials are summarized below.

Adler et. al.—Phase II Study—Solid Tumors—276 Patients (1984): (Adler et. al.—entire abstract): “Anguidine, a phase II agent, was used to treat 276 patients with solid tumors. The overall evaluability rate was 68%. Hematologic toxicity was substantial but not prohibitive. There were no complete responses, two partial responses, and 12 stabilizations.”

Bukowski et. al.—Phase II Study—Gastrointestinal Malignancies—134 Patients (1982) (Bukowski et. al.—entire abstract): “The Southwest Oncology Group conducted a phase II study of anguidine in 134 patients with gastrointestinal malignancies. Anguidine was administered as a 4-hour infusion at doses of 3.0 and 4.5 mg/m2 daily×5. Response rates for patients with colon carcinoma were 22% (four of 18 patients without previous chemotherapy) and 6% (four of 63 patients with previous chemotherapy). There were no responses in patients with pancreatic cancer (four patients) or gastric cancer (six). Toxic effects included thrombocytopenia (19.8%), leukopenia (18.8%), nausea and vomiting (49%), hypotension (37%), and confusion (12%). Antitumor activity of anguidine in patients with colon cancer may be similar to that of 5-FU, but nonhematologic toxicity is substantial.”

### Antiviral Activity:

Viruses require use of host ribosomes for their replication and spread. Inhibiting function of host ribosomes inhibits synthesis of viral proteins and hence inhibits viral replication and spread.

The antiviral activity of DAS was demonstrated in the 1980's by Okazaki et. al. at the Kochi Medical School, Japan (Okazaki et. al., “Inhibition by Trichothecenes of Replication of Herpes Simplex Virus Type 2”, Agric. Biol. Chem. 52 (3), 795-801, 1988). The study administered various concentrations of DAS (and 2 other Type A trichothecenes, T-2 and NEOS) to cultured human epidermoid cells (HEp-2) infected with Herpes Simplex Virus Type 2 (HSV2). The major findings:

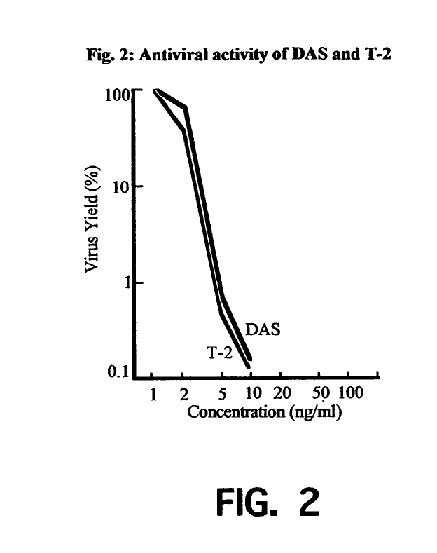

1) Baseline Safety—None of the compounds (DAS, T-2, NEOS) decreased the viability of HEp-2 cells at concentrations below 200 ng/ml for 3 days.
2) Efficacy—DAS and T-2 reduced virus yield to <1% by concentrations of 5 ng/ml as shown in FIG. 2 (from Okazaki, FIG. 2, p 797).
3) Best Results—The study found the best results were obtained when the drug was administered early in the viral replication cycle (i.e. newly infected cells).
4) Early Stage Viral Protein Synthesis and DNA Inhibition—The study demonstrated that “trichothecenes inhibited syntheses of HSV-2 early proteins and viral DNA” and “ . . . inhibition of early viral polypeptides by the toxin resulted in the decrease of viral infectivity and the inhibition of virus progeny DNA synthesis was a secondary effect of the toxins because of the reduced synthesis of one of the early peptides . . . probably HSV-2 DNA polymerase.”
5) Late Stage Viral Protein Synthesis Inhibition—“In this study, we also demonstrated that the toxins inhibited the virus replication by blocking late viral protein synthesis in the stage after early viral proteins . . . ”

Other studies, using other trichothecenes, and both HSV-1 and HSV-2, also demonstrated the anti-viral activity of trichothecenes, exactly as would be expected based on their MOA or preventing translation of RNA into viral proteins. Okazaki et. al. demonstrated the antiviral activity of macrocyclic trichothecenes in 1989 (Okazaki et. al., “Antiviral Activity of Macrocyclic Trichothecenes and Related Compounds Baccharinoids B-4 and B-5 against Herpes Simplex Virus Type 2”, Agric. Biol. Chem. 53 (5), 1441-1443, 1989) and Tani et. al. demonstrated the antiviral activity of trichothecenes against Herpes simplex virus types 1 and 2 in 1995 (Tani et. al., “Antiviral Activity of Trichothecene Mycotoxins Deoxynivalenol, Fusarenon-X, and Nivalenol against Herpes Simplex Virus Types 1 and 2, Microbiol. Immunol., 39(8), 635-637, 1995).

The incredible potential of this type of MOA as a broad spectrum antiviral was lost on prior art, as the irrational stigma of “toxin”, coupled with many unexplained untoward effects, played a large part in the technology not being pursued to commercialization. This is discussed in detail later in this application, when the unexplained untoward effects are explained and the toxin issues are put in perspective.

### Anti-Inflammatory Activity:

Inflammation is a highly protein synthesis driven event, fueled in part by production of inflammatory proteins such as cytokines and leukotrienes by tissue resident mast cells, as well as by other immune system cells, both tissue resident and non tissue resident. Inhibiting synthesis of mediators of inflammation by tissue resident mast cells and/or macrophages inhibits inflammation.

Blaylock et. al. (Blaylock et. al., National Institute of Environmental Health Sciences/NIH and Taiho Pharmaceutical Co. of Japan, “Topical Application of T-2 Toxin Inhibits the Contact Hypersensitivity Response in BALB/c Mice”, Journal of Immunology, Vol. 150, 5135-5143, No. 11, Jun. 1, 1993) demonstrated the anti-inflammatory activity of RNAi/PSR in contact hypersensitive BALB/C mice using T-2 (which has a virtually identical PSR profile as DAS as shown in FIG. 2 and also has a very similar IC50 to DAS, across various human cell lines, as shown in Table 2).

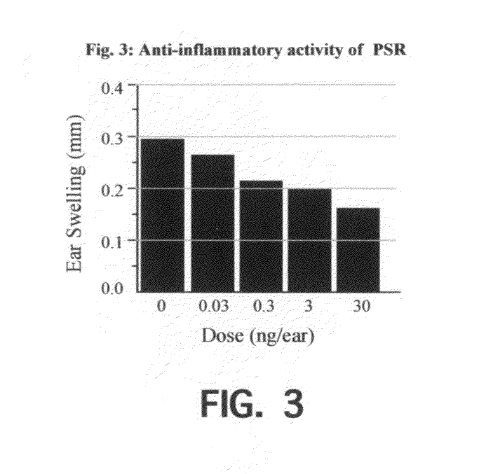

The study measured dose-response in oxazolone-sensitized and challenged mice. T-2 (dissolved in an ethanol/acetone/olive oil mixture) was topically applied immediately after application of the oxazolone challenge dose and ear thickness was assessed 24 hours later. The results are shown in FIG. 3 (excerpted from FIG. 1, pg. 5138, Blaylock et. al.). A 44% reduction in ear swelling was observed at the 30 ng topical dose.

### Human Systemic Safety:

The safety of DAS in humans was evaluated by Murphy et. al. in 1978 (Murphy et. al., “Phase I Clinical Evaluation of Anguidine”, Cancer Treat. Rep. 62: 1497-1502, 1978).

In the Murphy study of 39 patients, DAS (aka anguidine) was administered by iv over 30-60 minutes, daily for 5 days, every 2 weeks. The study found:

1) The No Observable Effect Level Dose (NOEL) dose was 2.4 mg/m2/day. “No drug-related toxicity was noted in any patient receiving <2.4 mg/m2/day×5 days of anguidine.” “No myelosuppression was seen at doses of <3 mg/m2”.
2) Symptoms at or above 3 mg/m2 for 25 patients (Murphy et. al, Table 3, p 1500):

| Symptoms | Percent of Patients |
|---|---|
| Nausea and vomiting | 100% |
| Myelosuppression | 79% |
| Hypotension | 20% |
| Somnolence (CNS incl. ataxia) | 20% |
| Diarrhea | 20% |
| Fever and chills | 12% |
| Generalized “burning” erythema | 12% |
| Stomatitis | 8% |
| Other | 12% |

3) Significance of Liver Function. “ . . . the severity of symptoms was found to correlate significantly with the presence or absence of liver metastases, elevated bilirubin levels, or liver function abnormalities”. DAS is primarily metabolized by the liver, and for patients with liver impairment a 3 mg/m2 dose was recommended.
4) Normal Therapeutic Dose (5 mg/m2/day×5)—resulted in the following myelosuppression (Murphy et. al. Table 5), as compared to the Normal Range (per Harrison's 15th ed. Table A-14 and 14th ed. Table A-8):

TABLE 5
Myelosuppression - Median Cell Count × 10³/mm³

| Course | No. of | Wbcs | | |
|---|---|---|---|---|
| No. | Patients | (Leukocytes) | Neutrophils | Platelets |
| 1 | 10 | 2.9 | 1.5 | |
| 2 | 10 | 2.9 | 1.8 | 76 |
| 3 | 3 | 2.9 | 1.7 | 135 |
| Normal Range | | (4.3-10.8) | (1.8-7.7) | (130-400) |

Other Phase I studies, at higher dose levels found:

Goodwin et. al.—Phase I Study (1978): When a 6 mg/m2 (QD X5) dose was administered by iv push: “CNS symptoms and hypotension were dose limiting but only mild to moderate myelosuppression was observed” and when administered by 8 hour infusion: “myelosuppression was unacceptable while other toxic manifestations were minimal.”

DeSimone et. al.—Phase I Study (1979): DeSimone tested doses ranging from 1.5 to 7.5 mg/m2 given as an infusion over 3 hours: “No myelosuppression was noted at any dose level. The toxic effects included nausea and vomiting, hypotension, CNS symptoms (confusion, hallucinations, and psychomotor seizures), chills, fever, and diarrhea. A dose of 5 mg/m2 of anguidine produced acceptable toxicity.”

The therapeutic doses used in representative examples of present invention range from several hundred times smaller to several million times smaller than the NOEL dose, as will be discussed in the individual reduction to practice examples, providing an extremely large margin of systemic safety.

### Method of Administration:

Topical: Preferred embodiment of current invention administers trichothecenes by topical application, mixed with either ethanol or methanol, however any suitable vehicle may be substituted. The latter serve no biological role other than to act a vehicle to facilitate uniform distribution of the trichothecene to a given area of skin. The advantage of a vehicle such as ethanol is that it self eliminates (i.e. evaporates) which precludes the vehicle from potentially interfering with deliver of therapeutic (i.e. trichothecene) over successive administrations (i.e. the vehicle does not clog the inter cellular spaces via which the therapeutic disperses). These mixtures are hereinafter referred to as “therapeutic compositions” or “pharmaceutical compositions” or “compositions” and nothing in this application is intended to limit trichothecene from being mixed with any suitable substance that may facilitate administration, uniformity of distribution, enhance absorption, increase efficacy, or with other trichothecenes or any other substances that serve any other beneficial purpose, the aforementioned combinations also called “therapeutic composition” or “pharmaceutical compositions” of present invention. The term “therapeutics” or “therapeutics of present invention” is generally intended to refer to the biologically active trichothecene(s). Numerous commercially available devices are available for administering therapeutic compositions as described above, some of which are listed by applicant in U.S. Pat. No. 6,355,251 col. 9, lines 20-51, incorporated herein by reference.

Inhalable: Numerous devices and excipients are commercially available for delivery of drugs by inhalation. Representative examples are listed by applicant in U.S. Pat. No. 7,012,091 col. 7, lines 15-32, incorporated herein by reference. As another example, Pfizer's dry powder inhaler, Spiriva HandiHaler, used to deliver Pfizer's anticholinergic COPD drug, could also be used for delivering DAS combined with a carrier/excipient such as lactose monohydrate, mannitol, or any other suitable carrier or vehicle. Individual doses are packaged in a hard gelatin capsule, which is inserted into the egg shaped inhaler, pierced, and inhaled from the inhaler. Other dry powder inhalers that may be substituted include commercially available dry powder inhaler devices such as the Pulmicort Turbuhaler breath activated dry powder inhaler (Astra USA Inc., Westborough, Mass.) or Galaxo Wellcome's Diskus inhaler. Alternatively, wet inhalers may also be substituted. There are many suitable commercially available inhaler devices and nebulizers. Aerosols produced by metered dose inhaler systems are about 2.5 to 2.8 μm in size upon entering the lungs and ˜50% of these aerosols are expected to deposit during normal tidal breathing. Devices are commercially available from sources such as Self Care, Emeryville, Calif., USA, Lumiscope Ultrasonic Nebulizer, the Dura-Neb® 3000 Portable compressor driven nebulizer, the PARI LC plus Nebulizer, the Omron CompAir Compressor Nebulizer System, the SpaceChamber™ aerosol spacer, AeroGen's (Mountain View, Calif.) OnQ® 1-2 micron particle generator for deep lung delivery, and other devices. Staccato™ by Alexza Molecular Delivery Corp. of Palo Alto, Calif. may also be used, which use heat to vaporize drugs for delivery by inhalation, based in part on observations of the cigarette as a very efficient drug delivery system.

### Dose Determination and Safety

### Dose Range—Efficacy, Safety, and Definitions

The stoichiometry of trichothecenes has been described in literature as one molecule per ribosome. The observed protein synthesis restriction profiles shown in FIG. 2 appear to be consistent with the described stoichiometry. The IC50 (50% inhibitory concentration) for DAS was 23 ng/ml in FIG. 2 and >99% protein synthesis inhibition was observed at 5 ng/ml, or at roughly double the IC50. The profile for T-2 followed a virtually identical pattern as shown in FIG. 2. The IC50 of DAS ranges from 1 ng/ml to 10 ng/ml depending on human cell line tested as shown in TABLE 3. Accordingly a concentration of 20 ng/ml of DAS would inhibit >99% of protein synthesis in all cell lines tested. Based on the stoichiometry and observed empirical data, a fairly linear dose/PSR relationship exists. In the least responsive cells tested, each 1 ng/ml increase in the concentration of DAS translates into a roughly 5% reduction in protein synthesis. The percentage decrease in protein synthesis per 1 ng/ml increase in concentration of DAS is much larger for more responsive cells such as human epidermoid cells. However, as a rule of thumb, it may be stated that each 1 ng/ml increase in concentration of DAS can be expected to result in at least a 5% reduction in translation of RNA into proteins (or conversely a 5% increase in RNAi or PSR).

Ex vivo, no reduction in cell viability was observed below 200 ng/ml for 3 days in monolayers of human epidermoid cells for either DAS or T-2. Maxwell et. al. found that deep dermal necrosis begins to occur in human skin at a topically applied dose of 1,400 ng/cm2 (Maxwell used a 35,000 ng/ml formulation to achieve this) for T-2. The highest concentrations used in representative examples are 200 ng/ml and highest topical doses administered are 24 ng/cm2 of skin, both of which are below any potential for causing cell death in the skin.

In vivo, in human clinical trials on 39 patients (Murphy et. al., p. 1499), the No Observable Effect Level (NOEL) dose for DAS by once daily rapid intravenous infusion for 5 consecutive days was 2.4 ng/m2, which translates into 4,100,000 ng for a 65 kg person (1.71 m2×2.4). Based on roughly 40 liters of blood and extracellular fluid, this translates into a roughly 100 ng/ml (4,100,000 ng÷40 liters) extracellular fluid concentration. No myelosuppression was observed below 3 mg/m2 per day, which translates into a 5,130,000 ng daily dose or 128 ng/ml extracellular concentration at which there is no reduction in cell counts. Therapeutic cancer doses of 5 mg/m2 (8,550,000 ng for a 65 kg person) translate into a 214 ng/ml extracellular fluid concentration and the reduction in cell counts observed was in rapidly proliferating cells, with blood counts at roughly 40% of normal as shown in Table 5. It should be noted that the reduction in blood cell counts at therapeutic dose levels could also be the result, in whole or in part, of cytostatic effect, as high turnover cell populations will drop in cell counts if they are not replenished. At doses of 6 mg/m2 administered by iv push, or 256 ng/ml extracellular concentrations, systemic neurological impairment symptoms began to manifest, indicating the start of systemically observable sulphydryl restriction (SHR) activity (discussed in further detail later in the application). Accordingly, the concentrations used in skin and lungs by present invention range from 10 ng/ml to 200 ng/ml, which are below any potential cellular cytotoxic effect in the tissues targeted for treatment (i.e. skin or lungs) based on both ex-vivo or in-vivo data in humans. The concentrations are also below any observable SHR activity levels. Schedules used in application include twice daily administrations for sustained efficacy for up to 3 days or daily administration for up to 5 days as theses schedules are not cytotoxic to skin or lungs based on the ex-vivo or in-vivo data from above (however any suitable schedules may be substituted).

The dose levels used in present invention are also well below the dermal irritancy levels. Hayes et. al. determined the median irritant concentration or ID50 (based on in vivo animal models) for DAS as 11,000 ng/ml with 95% confidence limits from 7,900 ng/ml to 15,400 ng/ml and for T-2 as 11,000 ng/ml with 95% confidence limits from 8,100 ng/ml to 15,000 ng/ml (from page 617, Table 1 of Hayes et. al. “Quantitative and Morphological Aspect of Cutaneous Irritation by Trichothecene Mycotoxins”, Western College of Veterinary Medicine, Saskatoon, Canada, Jun. 6, 1979). Accordingly, the maximum concentration of 200 ng/ml used in present invention is 55 times smaller than the ID50, well below the potential for any meaningful irritancy risk.

The therapeutic dose is calculated as the amount required to be delivered to a targeted tissue mass to achieve a desired concentration trichothecene (in ng/ml). Administered topical doses are generally larger than the therapeutic doses in order to compensate for less than 100% of the therapeutic dose being retained in the desired layer of skin. Administered inhalable doses are larger than the therapeutic dose in order to compensate for less than 100% of the therapeutic dose being retained by deposition in the lungs (i.e. the exact percentage depends on the drug−excipient particle's effective aerodynamic diameter).

The adjustment from therapeutic doses to administered doses, as shown in the representative examples, are computed based on the following for skin and lungs.

### Dose Determination—Skin:

The epidermis is made of columnar stacks of cells. The outermost layer (stratum corneum) is composed of a roughly 10 cell deep layer of dead keratinized squames, which rests on a layer of living epidermis around 10 cells deep. The only actively cycling cells in the epidermis are in the single cell thick basal layer. Once a basal cell divides it progresses through a maturation process that eventually transforms it into a squame. The epidermis is also interspersed with pigment cells (melanocytes) and macrophage like cells (langerhans cells). The epidermis does not contain blood vessels. Normal epidermis can be up to 0.2 mm thick. The stratum corneum can be up to 0.05 mm thick and the living epidermis can be up to 0.150 mm thick. The highest range of epidermal thickness is used in present application to insure the desired concentrations are reached in even the deepest parts of the epidermis.

The next layer down, the dermis, is composed primarily of connective tissue interspersed with cells including fibroblasts, mast cells, macrophages, endothelial (blood vessel) cells, and lymphocytes. The dermis is approximately 1 mm thick.

Below the dermis is a layer of subcutaneous fat which is more than 1 mm thick.

For purposes of present invention, the primary tissue targeted for treatment of viral infections or inhibition of epidermal hyperproliferation is the epidermis. The primary tissue targeted for anti-inflammatory activity is the dermis.

Type A trichothecenes such as DAS and T-2 have very high dermal retention rates in human skin by topical application in methanol or ethanol. At a topical dose of 79 ng/cm2, 97.2% of DAS was retained by the skin and 99.4% of T-2 was retained in the skin (from p. 381, table 1 of B. W. Kemppainen et. al, “Comparison of Penetration and Metabolism of Diacetoxyscirpenol, Verrucarin A, and T-2 Toxin in Skin”, Fd. Chem. Toxic., Vol. 25, No. 5, pp. 379-386, 1987).

Lipophilic molecules tend to distribute fairly homogeneously in the skin up until a certain concentration, after which steep concentration gradients start to form in the uppermost layers of the skin (as high concentrations clog the intercellular spaces, flow is restricted, and greater internalization occurs in the slow flow areas). Accordingly, where possible, doses closest to those used in present invention are used. The dose distribution of T-2 in human skin, dissolved in ethanol, and topically applied to the skin, was characterized by Maxwell et. al. (Maxwell et. al., “The In Vitro Penetration and Distribution of T-2 Toxin Through Human Skin”, Toxicology, 40, 1986, 59-74). At the lowest dose tested of 1,000 ng/cm2, approximately 38% of the total recovered dose was localized in the stratum corneum, 39% in the living epidermis layer, and 22% in the dermis. Adjusting slightly for the 97% DAS dermal retention rate, a dose distribution of 38% in living epidermis and 21% in dermis is used for purposes of present invention in calculating the amount of a topical dose required to achieve a desired therapeutic concentration in a given layer. From the Maxwell study, as dosing is increased above the 1,000 ng/cm2 level, concentration gradients begin to form with greater concentrations retained in the uppermost layers of the epidermis. However, since doses of present invention are in the 10 ng/ml-200 ng/ml range, well below the steep concentration gradient formation range, the dose distribution percentages as described above are used in dosaging computations (however any more suitable percentages may be substituted as they become available).

Based on the above dose distribution information, if the living epidermal layer is the targeted tissue mass, roughly 38% of a topically applied dose would be expected to localize in this layer. Accordingly, the topically applied dose would need to be 2.63 times higher than the desired dose (i.e. 1÷0.38) to achieve the desired tissue resident therapeutic dose in the living layer of the epidermis. If the dermal layer is the targeted tissue mass, the topically administered dose would need to be roughly 5 times the therapeutic dose (i.e. 1÷0.21=4.76).

As an example, if the living epidermal layer is the targeted tissue mass, the topically administered dose required to achieve a tissue resident therapeutic dose in this layer would be computed as follows. The thickness of the living epidermal layer is 0.15 mm. The volume of living epidermis per square centimeter of skin surface area is thus 15 cubic mm (10 mm×10 mm×0.15 mm). Converting this to ml at a rate of 1 cc=1 ml (or 1 cubic mm=1/1000 ml) gives the respective volume in ml as 0.015 ml of living epidermis per square cm of skin surface area (15 cubic mm×0.001 ml/cubic mm). At 0.015 ml living epidermal volume per square centimeter for normal skin, to achieve a 10 ng/ml tissue resident dose concentration requires a 0.15 ng dose (i.e. 10 ng/ml×0.015 ml) per square centimeter of skin. Adjusting upward to compensate for the 38% dose localization rate in the living epidermal layer translates to 0.39 ng administered dose required per square centimeter (i.e. 2.63×0.15 ng).

The dermis is around 1 mm thick, and accordingly a square centimeter of skin would have an underling dermis volume of around 100 cubic mm (i.e. 10 mm×10 mm×1 mm=100 mm³) or 0.1 ml (100 cubic mm×0.001 ml/cubic mm=0.1 ml). A 10 ng/ml dermis resident dose translates into 1 ng (10 ng/ml×0.1 ml=1 ng) which in turn would require a 5 ng topically applied dose based on the skin distribution data (i.e. only 21% of a topically applied dose winds up localizing in the dermis).

Dose Determination—Lungs:

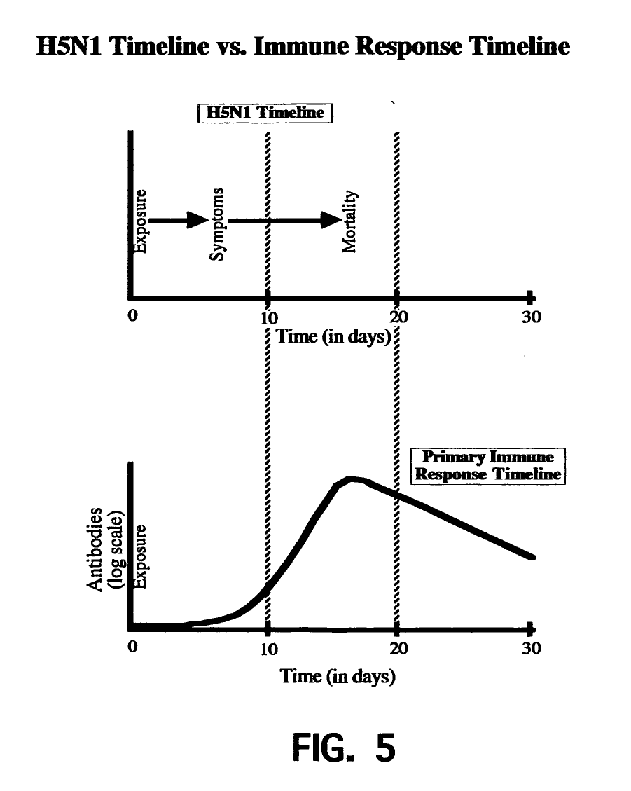

The primary adjustment for inhaled lung doses relates to the rates of deposition of drug in the lung. This in turn is directly dependent on the effective aerodynamic diameter of the eventual microparticularized particle size used (i.e. drug+excipient). The dosimetry of inhaled particles is shown in FIG. 5. In general, if particle size is too small, most of the dose gets exhaled and if the particle size is too big, most of the dose winds up in the back of the throat. As shown in FIG. 5, a 6 μm (micron) particle size favors tracheobronchial deposition with roughly 35% of the dose deposited in the tracheobronchial tissue and 20% in alveolar tissue. As shown in FIG. 5, a 3 μm particle favors alveolar deposition with 40% of the dose deposited in the alveolar tissue and 17% deposited in tracheobronchial tissue. If homogenous tracheobronchial and alveolar deposition are desired, a 5 μm particle is a good choice, with roughly one third of the inhaled dose depositing on tracheobronchial tissue and one third depositing on alveolar surfaces.

As an example, if equal tracheobronchial and alveolar deposition is desired, a 5 μm microparticularized inhaled particle could be used, with the dose administered by inhalation being 50% greater than the therapeutic tissue resident dose desired (i.e. as only two thirds of the inhaled dose winds up being deposited in the lungs). The average human lungs are around 1,200 grams or 1.2 liters. As an example, if a 10 ng/ml tissue resident concentration is desired, the total therapeutic lung dose would be 12,000 ng (i.e. 10 ng/ml×1,200 ml=12,000 ng) and accordingly the dose administered by inhalation (i.e. loaded into the inhaler) would be 18,000 ng (i.e. 1.5×12,000) in order to achieve the desired therapeutic tissue resident concentration of 10 ng/ml in the lungs.

### Systemic Safety Computations:

Because of the Phase I human data obtained for DAS, the doses used in present invention can also be compared against the human systemic NOEL (no observable effect level dose). As previously disclosed, the human NOEL dose for DAS was established in humans as 2.4 mg/m2 per day. For a 65 kg human with a 1.71 m2 BSA the NOEL dose translates into a 4,100,000 ng daily dose (i.e. 2.4 mg/m2×1.71 m2=4.1 mg or 4,100,000 ng). No myelosuppression was observed below 3 mg/m2 per day, which translates into a 5,130,000 ng daily dose. The recommended therapeutic cancer dose of 5 mg/m2 per day translates into a 8,550,000 ng daily dose.

Using the systemic NOEL dose level, if 1 square centimeter of epidermis is treated with a 10 ng/ml concentration of DAS in the living epidermis layer (i.e. 0.39 ng administered dose, as previously computed), >97% of the dose would be expected to be retained in the skin, however, assuming somehow 100% of the administered dose accidentally wound up directly in the bloodstream, this would be 10 million times smaller than the daily NOEL dose (i.e. 4,100,000 ng÷039 ng=10,512,821). Accordingly, there would be absolutely on effect on the patient (the amount would be readily metabolized by the liver or so dilute systemically as to have no observable impact on any cells it internalized into, and where it would be intracellularly metabolized in about 12 hours anyway).

Likewise, if the 12,000 ng therapeutic lung dose of DAS computed above went directly to blood with 0% lung retention, this would still be 342 smaller than the NOEL dose (i.e. 4,100,000÷12,000). If the entire 18,000 ng dose packaged lung administration (e.g. gelatin capsule containing drug+excipient to be loaded into the inhaler) was somehow inadvertently dissolved in solution and injected directly into the blood, this would still be 228 times smaller than the NOEL dose (i.e. 4,100,000÷18,000).

Even under the extreme abuse situations, the formulations are still very safe. The average human has ˜18 square feet of skin (17,000 sq. cm.). If a patient were to apply the 039 ng/cm2 epidermal dose to all 17,000 square centimeters of skin, that would translate to 6,630 ng dose of DAS (i.e. 0.39×17,000=6,630). Once again, unrealistically assuming 100% goes directly to blood instead of 97% being dermally retained as expected, the full body administered dose is still 618 times smaller than the systemic NOEL dose (i.e. 4,100,000÷6,630=618). The patient would have no ill effects.

### Reduction to Practice Examples

Examples by disease condition are provided to give further guidance on methods of use of compositions of present invention as discussed above.

### Example 1

### Herpes Lesions

Oral and Genital Herpes lesions are caused by the Herpes Simplex Virus (HSV). HSV is a cytolytic (cell destructive) virus that causes cell necrosis with resulting inflammation.

Prior Art: Prior art prescription treatments for oral herpes lesions include penciclovir cream (Denavir from Novartis) and acyclovir (Zovirax from GlaxoSmith Kline).

Per the full prescribing information, Denavir cream contains 10 mg of penciclovir per gram. Penciclovir inhibits HSV polymerase and hence inhibits HSV DNA synthesis. The cream is to be administered at the earliest sign of an oral herpes lesion (i.e. tingling, redness, itching, or bump). The cream is to be applied every 2 hours during waking hours for 4 days. Mean duration of lesions was one-half day shorter in subjects treated with Denavir versus placebo (i.e. 4.5 days versus 5 days).

Per the full prescribing information, Zovirax cream contains 50 mg of acyclovir pre gram. Acyclovir is a synthetic nucleoside analogue that inhibits herpes viruses. It is to be administered within 1 hour of noticing signs or symptoms, administered 5 times per day, with continued treatment for 4 days. The median duration of recurrent oral herpes episodes were one half day shorter for treated versus untreated patients (i.e. 4.5 days versus 5 days).

### PRESENT INVENTION

### Example 1a

### Early Symptom Treatment, Epidermis Antiviral IC100, Dermis Anti-Inflammatory>IC4%

Under present invention, at the earliest sign of symptoms (i.e. tingling, itching), when no increase in epidermal volume had yet occurred, a 0.39 ng dose of DAS would be applied (to the 1 square centimeter of skin where the tingling, redness, or itching was manifesting) twice daily for up to 3 consecutive days. As previously disclosed, 0.39 ng is the administered dose that would need to be applied to achieve a 10 ng/ml concentration in the living epidermal layer. The cells in this layer have ribosomes that are usurped by the virus for its replication and spread. However, any suitable tissue depth targeting, dose, or administration schedule may be substituted for the above.

Rationale: At 10 ng/ml, ˜100% viral inhibition in human epidermoid cells is observed, as shown in FIG. 2. The anti-inflammatory benefit at this level would be modest. At a 0.1 ml dermis volume per square centimeter of skin and roughly 20% of the dose localizing in the dermis, a concentration of roughly 0.78 ng/ml would be achieved in the dermis (i.e. 039 ng×0.2=0.078 ng in the dermis 0.1 ml dermis volume=0.78 ng/ml). Based on the previously disclosed rule of thumb of 1 ng/ml for every 5% increase in protein synthesis restriction, the 0.78 ng/ml dermal concentration would translate into at least a 4% inhibition in synthesis of protein mediators of inflammation by mast cells and macrophages of the dermis.

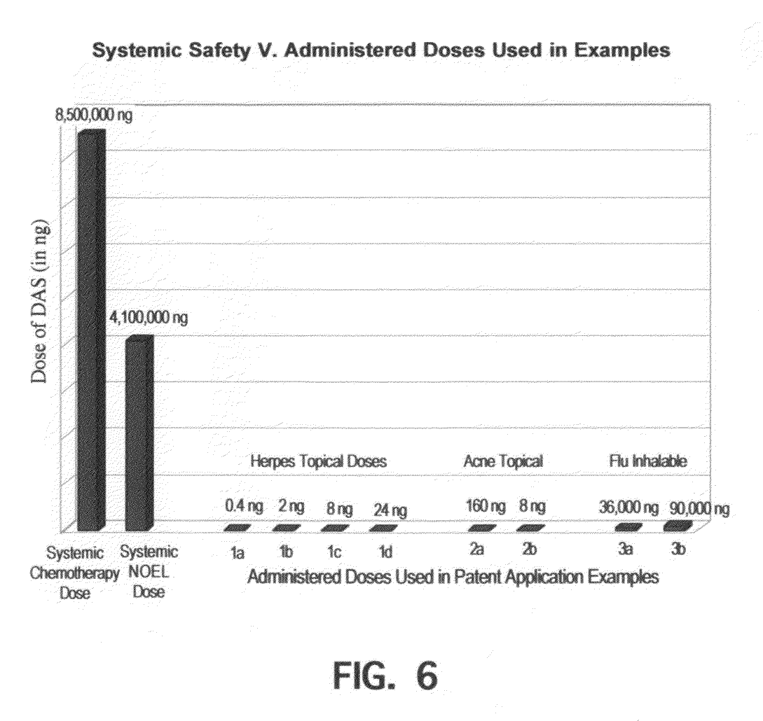

Safety: As previously disclosed, the entire administered dose of 039 ng is 10 million times smaller than the systemic NOEL dose and as shown in FIG. 6.

### Example 1b

### Early Symptoms—Epidermis Antiviral IC100, Dermis Anti-Inflammatory>IC20

As an example, an higher strength herpes lesion formulation may be provided for full viral inhibition plus more potent anti-inflammatory activity. Using a 50 ng/ml dose would provide both complete viral inhibition in the living epidermal layer and boost the concentrations in the dermis where inflammatory cells are located. At 0.015 ml epidermal volume per square centimeter for living epidermis, to achieve a 50 ng/ml tissue resident dose concentration requires a 0.75 ng dose (i.e. 50 ng/ml×0.015 ml) per square centimeter of skin. Adjusting upward to compensate for the 38% dose localization rate in the living epidermal layer translates to 2 ng administered dose required per square centimeter. The dose would be applied twice daily for 3 days “on” one day “off” or alternatively could be applied once daily for 5 consecutive days or any other suitable schedule may be substituted. The 2 ng topically administered dose also translates into a 4 ng/ml concentration in the dermis (i.e. 2 ng×0.2=0.4 ng in the dermis÷0.1 ml dermis volume=4 ng/ml). Based on our rule of thumb, this would inhibit at least 20% of synthesis of protein mediators of inflammation by mast cells and macrophages located in the dermis.

Rationale: The 2 ng topical dose translates into a 50 ng/ml concentration in the living epidermal layer and a 4 ng/ml concentration in the dermis. The 50 ng/ml is more than adequate to achieve 100% viral inhibition in the living layer of the epidermis. The 4 ng/ml concentration in the dermis would inhibit at least 20% of de novo synthesis of protein mediators of inflammation by mast cells and macrophages located in the dermis (based on the previously disclosed rule of thumb that each 1 ng/ml increase in concentration yields at least a 5% reduction in protein synthesis in all human cell lines tested). The anti-inflammatory effect is also consistent with the observed mouse model data, where a 3 ng/ear topical dose yielded a roughly 30% reduction in inflammation as shown in FIG. 3. The inference is that the percentage inhibition of synthesis of mediators of inflammation correlates fairly well to the percentage reduction in inflammation observed at this dose level. The expectation would also be that early administration would yield the best results, as therapeutics of present invention prevent de novo synthesis of protein mediators of inflammation, but have no mechanism to inactivate mediators of inflammation that were synthesized prior to administration of therapeutics of present invention.

Safety: Based on a treatment area of around 1 square centimeter of skin, a 2 ng dose is 2 million times smaller than the daily NOEL dose (i.e. 4,100,000 ng÷2=2,050,000), assuming 0% is dermally retained and 100% of the dose goes directly to blood. This is an extremely large safety margin and as shown in FIG. 6.

### Example 1c

### Full Viral Inhibition (IC100)/Maximum Anti-Inflammatory Activity (>IC80)

In the absence a trans epidermal delivery system, the largest concentration that could be used, without causing a potential for reduction of cell viability in the living epidermal layer, is 200 ng/ml. This is four times greater than the concentration used in example 1b. This translates into a 8 ng topical dose per square centimeter of skin, which in turn translates to a dermis layer concentration of 16 ng/ml ((i.e. 8 ng×0.2=1.6 ng in the dermis÷0.1 ml dermis volume=16 ng/ml), which in turn translates into at least an IC80 in the dermis.

It should be noted that the anti-inflammatory IC80 relates to inhibition of de novo synthesis of protein mediators of inflammation and would not necessarily translate into a 80% reduction in inflammation. The animal model data suggest that full inhibition of de novo synthesis may only translate into a maximum reduction in observed inflammation of 44% as shown in FIG. 3. Mast cells store preformed mediators of inflammation, which are released upon mast cell activation (e.g. preformed compounds include histamine, heparin, tryptase, kallikrein, eosinophil chemotactic factor, neutrophil chemotactic factor) and PSR would not be expected to inhibit the release of these compound (just their re-synthesis). Mast cells also newly synthesize and release mediators of inflammation upon activation (e.g. newly synthesized compounds include cytokines, leukotrienes, thromboxane, and platelet activating factor) which would be inhibited by PSR. Furthermore, numerous other immune system cells, not all of which are tissue resident, are also involved in mediating inflammation. In vivo animal model data (FIG. 3) suggests that PSR's contribution of inhibition of de novo synthesis of mediators of inflammation in tissue resident mast cells and macrophages may be limited to an observed reduction in inflammation of around 44%.

Safety: The 8 ng topical dose is half a million times smaller than the daily systemic NOEL dose (i.e. 4,1000,000 ng÷8 ng=512,500) and as shown in FIG. 6.

### Example 1d

### Later Stage Lesion

If treatment is not initiated until after the lesion had become inflamed, the dose could be adjusted for the increase in volume. As an example, if the normal thickness of the living layer of the epidermis plus dermis of 1.150 mm had now swollen to 3.45 mm, or three times its normal thickness, the doses would be adjusted as follows. To achieve a 50 ng/ml dose in the swollen living epidermis would now require a 2.25 ng therapeutic dose (i.e. 50 ng/ml×0.045 ml) per square centimeter of skin, which adjusted upward for the 38% layer specific retention rate would translate into a 6 ng topical dose per square centimeter of skin. If the maximum 200 ng/ml living epidermal layer concentration is used, a topical dose of 24 ng is used, which results in a 16 ng/ml dermis layer concentration (24 ng×20%=4.8 ng dermis layer concentration÷0.3 ml=16 ng/ml) or an IC80 in the dermis.

Safety: The 6 ng dose is 680,000 times smaller than the daily systemic NOEL dose (i.e. 4,100,000 ng÷6=683,333), assuming 0% is dermally retained and 100% of the dose goes directly to blood. The antiviral IC100/anti-inflammatory IC80 dose of 24 ng is 170,000 times smaller than the daily systemic NOEL dose (4,100,000÷24=170,833), assuming 0% is dermally retained and 100% of the dose goes directly to blood, and as shown in FIG. 6.

Related Patent Disclosure: The above schedule has deliberately been set to avoid any cytotoxic effects, and only to provide dual action antiviral and anti-inflammatory activity. As previously disclosed, no reduction in human epidermoid cell viability was observed at 200 ng/ml over three days, and concentrations used in instant application are at or below this concentrations. The primary reason for doing this is to avoid a potential double patenting issue. Applicant's U.S. Pat. No. 6,355,251 teaches use of trichothecene for epidermal chemexfoliation and uses cytotoxic doses and schedules. Epidermal exfoliation could be a viable alternative to treating herpes lesions—an approach that would basically kill all the virally infected cells and jettison the infected patch of skin to avoid a painful T-Cell mediated immune response and to allow the start of regrowth by non infected cells from the periphery to fill the void. However, such an approach would be covered under applicant's U.S. Pat. No. 6,355,251 for epidermal exfoliation, and as such, only non cytotoxic doses are used in both the specifications and the related claims of instant invention.

The above schedules are not optimal or absolute. They are presented only to fulfill the reduction to practice requirement of a efficacious, safe examples and are not intended to limit the scope of the application and its related claims in any way. Innumerable variants of the above doses, schedules, and trichothecene used are possible and may be substituted for the above.

Summary of Advantages over Prior Art: Present invention's use of trichothecenes such as DAS provides a novel mechanism of action over prior art methods. Present invention's approach of targeting host ribosomes provides full spectrum inhibition of all viral proteins. This includes inhibition of HSV DNA polymerase, which in turn also inhibits synthesis of viral DNA. The dual action inhibition of tissue resident mast cell synthesis of protein mediators such as cytokines and leukotrienes, and hence the related inflammation, is also a large advantage, as it is the inflammation that is responsible for the pain and swelling in herpes lesions.

### Example 2

### Acne

Acne is an infection in the pore that houses a hair follicle. The hair follicle pore is basically an invagination of the epidermis that extends deep into the dermis with the root of the hair follicle at the bottom of the pore. Along the length of pore, structures called sebaceous glands produce an oily substance called sebum (lipid content of ˜41% triglycerides, 25% wax monoesters, 16% free fatty acids, 12% squalene).

The four key factors responsible for Acne Lesions (per eMedicine, Julie C. Harper MD article, “Acne Vulgaris, 2005 provided under IDS) are:
1) follicular epidermal hyperproliferation which plugs the follicle
2) excess sebum
3) presence and activity of P. Acnes bacteria
4) inflammation

Acne is caused by epidermal hyperproliferation (believed to be hormonal related) which plugs the sebaceous gland, causing excess sebum to accumulate, which provides an opportunistic environment for the P. Acnes bacteria to thrive in, which in turn results in inflammation.

Prior Art Treatments: Antibacterial—Topical antibiotics include erythromycin and clindamycin. Systemic antibiotics include tetracycline, minocycline, trimethoprim, and azithromycin. Antiproliferative/Anti-Inflammatory Agents—Topical retinoids include adapalene, tazarotene, and tretinoin (modulate gene expression). Systemic retinoids-isotretinoin (Accutane).

Present Invention's Treatment: The methods of present invention use trichothecenes in therapeutically effective amount to inhibit the epidermal hyperproliferation and inhibit inflammation. The antiproliferative doses and schedules used are cytostatic, not cytotoxic, in order to avoid a double patenting issue with applicant's patent U.S. Pat. No. 6,355,251 as disclosed in Example 1. As such, the antibacterial activity is obtained by depriving the bacteria of their food source as inhibiting the epidermal hyperproliferation prevents clogging of the follicle and accumulation of excess sebum on which the P. Acnes bacteria opportunistically thrives.

An example of both a facial prophylactic dose and an individual inflamed acne lesion “spot” dose are provided.

### Example 2a

### Facial Prophylactic Dose

For a teenage patient with persistent facial acne, a prophylactic dose would be applied to the entire facial area of ˜200 square centimeters. Any suitable delivery system could be used (e.g. roller-ball bottle, medicated wipes or pads, bottle with resident sponge applicator, etc. . . . ) to deliver a topical solution of trichothecene, which may also include a therapeutic dose of antibacterial agent.

A 20 ng/ml therapeutic dose of DAS could be used, applied twice daily, every second day. At 0.015 ml epidermal volume per square centimeter for normal skin, to achieve a 20 ng/ml tissue resident therapeutic concentration requires a 03 ng dose (i.e. 20 ng/ml×0.015 ml) per square centimeter of skin. Adjusting upward to compensate for the 38% localization rate in the living epidermal layer translates to 0.8 ng administered dose required per square centimeter. Based on a treatment area of around 200 square centimeters of facial skin a total dose of 160 ng would be applied, which is 25,000 thousand times smaller than the daily systemic NOEL dose (i.e. 4,100,000 ng÷ 160 ng=25,626), assuming 0% is dermally retained and 100% of the dose goes directly to blood. This is an extremely large margin of systemic safety as shown in FIG. 6.

Rationale: The 20 ng/ml epidermal concentration is the IC100, inhibiting virtually all protein synthesis, resulting in cytostatic effect as previously disclosed. The twice daily administration every second day effectively inhibits epidermal proliferation by 50%. Alternatively, once daily administration every day would also provide ˜50% inhibition. Hormonal spikes in teenage years can accelerate basal layer proliferation rates, and the purpose of the dosing schedule is to provide a 50% reduction of the hyper proliferated rate. The prevention of hyperproliferation prevents the clogging of the pore and accumulation of excess sebum on which P. acnes opportunistically thrive. The antibacterial activity is achieved by depriving the P. acnes bacteria of its food source and the opportunistic environment it needs to thrive in. The above dosing schedule is not absolute, as any suitable administration schedule may be chosen to set the basal cell proliferation schedule to any desired level. The 0.8 ng per square centimeter topical dose translates into a 1.7 ng/ml dermis concentration (at 21% dose distribution and 0.1 ml dermis volume) for a very low level of anti-inflammatory activity.

### Example 2b

### Individual Inflamed Acne Lesion Dose

Up to a 200 ng/ml living epidermal layer therapeutic concentration of DAS could be used for an inflamed acne lesion, applied twice daily, for up to 3 consecutive days (however any other suitable dose, trichothecene, or schedule may be substituted). To achieve a 200 ng/ml epidermal layer dose concentration requires a 3 ng therapeutic dose (i.e. 200 ng/ml×0.015 ml) or 8 ng topically administered dose (i.e. 3 ng 38%).

The administered trichothecene would have two targets—1) the epidermal layer for inhibition of epidermal hyperproliferation and 2) the reservoir of sebum/bacteria/immune cells in the pore for the anti-inflammatory activity. The epidermis surrounding the opening of the pore should follow the typical distribution patterns as previously disclosed, and as such the 200 ng/ml epidermal concentration is the IC100 antiproliferative dose. Using the standard dermis dose distribution of 21% of topical dose and 0.1 ml dermis volume translates into a 17 ng/ml concentration, or IC80 in the dermis surrounding the pore. Based on the previous discussion in Example 1d, inhibition of de novo synthesis of mediators of inflammation in this way could be expected to result in as much as a 44% reduction in observed swelling in the skin.

Computation of the amount of total reduction in size of inflamed the acne lesion would be more complicated because part of the lesion's mass is comprised of the clogged pore filled with sebum, bacteria, and immune system cells. The dose distribution in this part of the acne lesion has not been characterized. The opening of the pore would not have a stratum corneum layer or epidermal layer to penetrate. The opening of the pore would be closed only by the mechanical tension of two vertical layers of epidermis pressing against each other, and not tightly connected by intracellular adhesion molecules or gap junctions as would occur in normal epidermis. For an extremely small molecule such as DAS (366 MW) that readily slips between the 2-4 nm spacing between connected cells, the pore would be a fairly large access channel. However, since the pore is an invagination of the skin, a portion of the DAS flowing down the pore could be expected to localize in the invaginated epidermis with the remainder distributing into the sebum pool. Accordingly, the immediate anti-inflammatory IC may be more or less than IC80 in this region. The longer term skin thinning from inhibition of epidermal proliferation would undo the clog and release the sebum, bacteria, and immune cells reservoired in the pore, resulting in a fairly abrupt reduction in pimple size and inflammation.

Safety: A 8 ng dose is more than half a million times smaller than the daily systemic NOEL dose (i.e. 4,100,000 ng÷8=512,500), assuming 0% is dermally retained and 100% of the dose goes directly to blood, and as shown in FIG. 6.

Rationale: The IC100 epidermal antiproliferative concentration is designed to decisively start the process of skin thinning and inhibition of any further proliferation, for the purpose of unclogging the pore and releasing the reservoired sebum, bacteria, and immune cells. The dermis IC80 is used to inhibit de novo synthesis of mediators of inflammation by immune cells located in the dermis, as this is what is responsible in part for the redness, pain, and raised appearance.

Summary of Novelty Over Prior Art: The compositions and methods of present invention would provide a new therapeutic treatment option for acne, Compositions of present invention could also be used concurrent with anti-bacterial agents already in use to greatly expand the spectrum of anti acne activity (i.e. antiproliferative, antibacterial, anti-inflammatory). The mechanism of action (MOA) of present invention is perhaps most analogous to Retinoids, which also have antiproliferative and anti-inflammatory activity. Retinoids interfere with DNA expression. The entire purpose of DNA is to be translated into protein. Therefore, protein synthesis inhibition at the ribosomal level is also effectively interfering with DNA expression. The primary mechanistic difference is that as retinoids interfere with DNA/RNA transcription pathways at the earliest stages, trichothecenes interfere with RNA/Protein translation pathways at the latest stage (i.e. at the ribosome). The end point is effectively the same, however PSR has two mechanistic advantages. First, RNAi/PSR is much faster in obtaining the desired effect, as PSR chokes off protein synthesis at the very end of the pathway rather than at the very beginning like retinoids—which effectively require the dismantling of the existing pathway in order to reach the same eventual endpoint of protein synthesis inhibition. Second, the recovery from discontinuation of drug is also much faster with PSR, as the RNA is already available for immediate transcription into proteins, versus retinoids where the entire pathway needs to be rebuilt from the beginning.

### Example 3

### Lung Antiviral and Anti-inflammatory

Viruses lack the requisite genome to synthesize their own ribosomes and as such rely on host ribosomes to synthesize the proteins needed for their replication and spread. Trichothecenes such as DAS, which inhibit host ribosome function, would provide 3 exceptionally novel treatment attributes. First, they would be effective against any virus that required the use of host ribosomes, providing unprecedented broad spectrum antiviral activity. Second, the treatment method would be highly “mutation resistant” as any mutated viral proteins would still need to be synthesized on the host ribosomes and hence the virus would still be inhibited. Third, the method of present invention would also provide an unprecedented solution for viral lung infections that had accompanying life threatening inflammation such as Avian Flu (H5N1) or Spanish Flu.

An inhalable Antiviral and Anti-inflammatory that targets the respiratory tract has exquisite significance to the H5N1 strain of Avian flu.

Background: Influenza requires use of host ribosomes to synthesize the proteins required for its replication and spread. The influenza family contains five genera, classified by variations in nucleoprotein antigens: influenza A, influenza B, influenza C, thogotovirus, and isavirus. Influenza A are a major cause of influenza in humans and all the past influenza pandemics in humans have been caused by influenza A viruses. The genome consists of 10 genes located on eight separate negative-sense single-stranded RNA molecules; this allows for genetic reassortment in single cells infected with more than one virus and may result in multiple strains that are different from the initial ones. Viral RNA molecules are translated on host ribosomes into the following ten proteins: three transcriptases (PB2, PB 1, and PA), two surface glycoproteins (hemagglutinin [HA] and neuraminidase [NA]), two matrix proteins (M1 and M2), one nucleocapsid protein (NP), and two nonstructural proteins (NS1 and NS2).

From a human pathogenicity standpoint, the three most important RNA molecules (and the resultant proteins translated from those RNA molecules) are HA, PB1, and NS1.

HA is the surface glycoprotein used by the virus to enter a cell (NA is used to exit the cell). Avian influenza HA binds to alpha 2-3 sialic acid receptors on cell surfaces while human influenza HA binds to alpha 2-6 sialic acid receptors. Humans have avian type receptors at very low density. HA mutations at positions 182, 192, 223, 226, or 228 have shown altered binding attributes to avian and/or human surface receptors. These are the types of mutations that can change avian flu into a human pandemic strain.

PB 1 RNA codes for a viral polymerase molecule that is especially important in virulence. PB 1 codes for the PB 1 protein and the PB 1-F2 protein. One amino acid change in the PB 1-F2 sequence at position 66 was found to correlate with pathogenicity in the 1997 H5N1 outbreak in Kong Kong.

NS codes for two proteins NS1 and NEP. NS was correlated with pathogenicity of the H5N1/97 virus. NS1 is involved in RNA transport, splicing, and translation. A single amino acid change at position 92 of NS1 greatly increased the pathogenicity of the H5N1 influenza virus. NS1 is believed to be responsible for an enhanced proinflammatory cytokine response (especially TNF-alpha) in human macrophages.

Human mortality from recent H5N1 outbreaks has ranged from 60-80%. Immune response-mediated inflammation is a key cause of mortality in Avian flu in what has been termed “the cytokine storm”. H5N1 is known to involve a primary immune response. The human immune system takes 7-10 days post-exposure to initiate a primary immune response (as measured by antibody production), which peaks at 15-20 days post exposure. The World Health Organization (WHO) uses a 7 day incubation period for H5N1 (i.e. time from exposure to symptoms) and mortality occurs 8-12 days after symptoms, which puts mortality at around 17 days after exposure, or right at the peak of the immune response. The two timelines are shown in FIG. 5 for reference as they are used in part in administration schedule determination.

Prior Art: Flu drugs Tamiflu and Relenza work by inhibiting some strains of neuraminidase. They have yet to be fully tested or proven in a H5N1 pandemic situation.

Present Invention: Preferred embodiment of present invention uses DAS in therapeutically effective amounts to inhibit both viral replication and inflammation. DAS is used to inhibit RNA translation for all 10 viral proteins. This not only includes inhibiting synthesis of any strain of neuraminidase, but inhibiting synthesis of HA, PB 1-F2, and NS1. DAS would also inhibit tissue resident mast cell and macrophage production of protein mediators of inflammation such as cytokines and leukotrienes.

The dosing and scheduling would be dependent in part on where a patient is on the H5N1 timeline.

### Example 3a

### Early Infection Dosing Example

A healthcare worker the was exposed for 2 days to a group of symptomatic patients before it was ascertained that the outbreak was H5N1 would be a representative example of an early exposure case.

Present invention would use a 20 ng/ml therapeutic dose of DAS (however any other suitable dose or trichothecene may be substituted), which would be ≧IC99 for all of the human cell lines tested. This would provide roughly 100% ribosomal inactivation and hence 100% viral protein synthesis inhibition, as shown in FIG. 2. It would also provide a IC99 level inhibition of tissue resident mast and macrophage cell synthesis of mediators of inflammation such as cytokines and leukotrienes, which can cut the level of inflammation by up to 44%, as shown in FIG. 3.

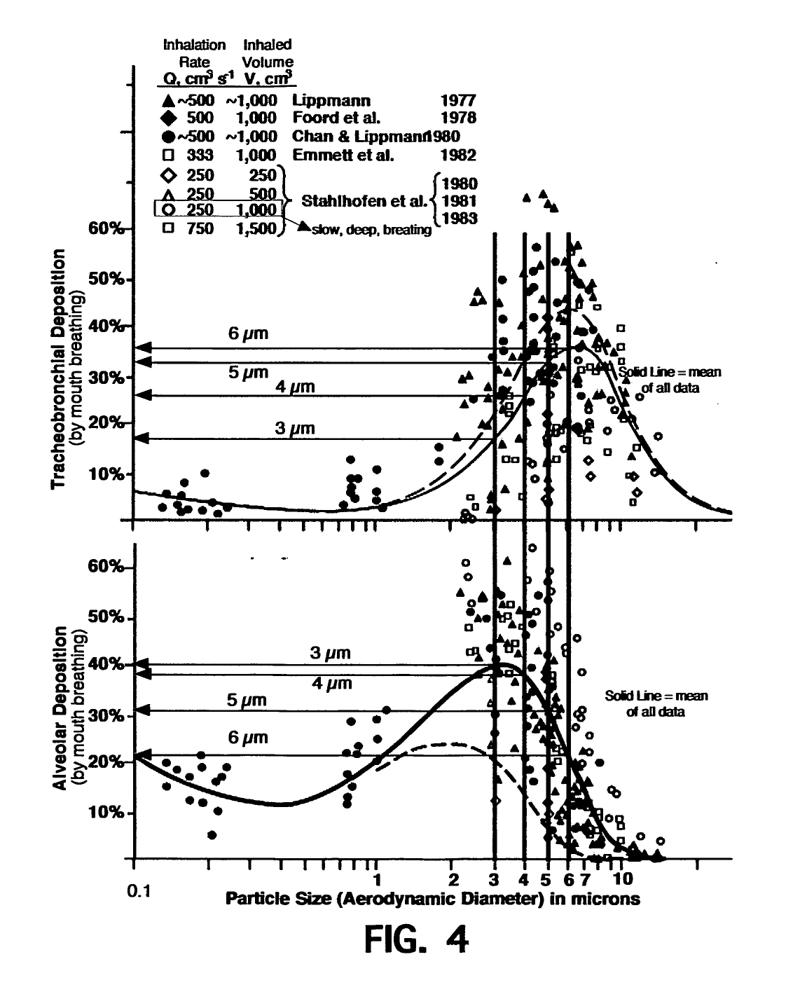

The administered dose, or dose to be loaded into the inhaler, would be computed by methods previously described. In the 20 ng/ml example above, assuming a 5 μm microparticularized inhaled particle is be used to achieve equal tracheobronchial and alveolar deposition, the dose administered by inhalation would need to be being 50% greater than the therapeutic tissue resident dose desired (i.e. as only two thirds of the inhaled dose winds up being deposited in the lungs as shown in FIG. 4). The average human lungs are around 1,200 grams or 1.2 liters. If a 20 ng/ml tissue resident concentration is desired, the total therapeutic lung dose would be 24,000 ng (i.e. 20 ng/ml×1,200 ml=24,000 ng) and accordingly the dose administered by inhalation (i.e. loaded into the inhaler) would be 36,000 ng (i.e. 1.5×24,000) in order to achieve the desired therapeutic tissue resident concentration of 20 ng/ml in the lungs.

Safety: From a worst case safety perspective, assuming 100% of the administered dose went directly to blood, this would be 114 times smaller than the NOEL dose, and the patient should have no observable ill effects (i.e. 4,100,000 ng÷36,000=114) and as shown in FIG. 6.

A representative dosing schedule could be twice daily administration for 3 days on and 1 day off for 4 cycles. This would stretch out the H5N1 viral proliferation timeline shown in FIG. 5 roughly 3 fold, greatly reducing the number of virally infected cells at any point in time relative to the primary immune response curve (i.e. cuts the level of virally infected cells to roughly 30% of the level that would be expected in the absence of treatment). The objective is to reduce the percentage of infected cells to as few as possible up until the point a full blown immune response has been established. Inhibiting viral replication and spread limits the number of infected cells that eventually need to be killed by cytotoxic T-Cells, which in turn limits morbidity and mortality. Furthermore, the second MOA of inhibiting tissue resident mast cells and macrophages from producing cytokines and leukotrienes would function directly to prevent mortality from the “cytokine storm”.

An alternative dosing schedule could be once daily administration for 15 days. This would effectively mean the drug would work for 12 hours per day, effectively providing a 50% reduction in the number of virally infected cells that would exist at the time a full blown immune response had been established. Once again this would function to limit the number of infected cell that eventually needed to be killed as well as help suppress inflammation.

### Example 3b

### Late Infection Dosing Example

A person that presents with early symptoms would be roughly 7 days post exposure based on the WHO guidelines and as shown in FIG. 5. Symptoms coincide with the beginning of a primary immune response as shown in the two juxtaposed timelines in FIG. 5. As a representative example, a 50 ng/ml concentration could be used, administered twice daily, for 3 days on, one day off, for up to 3 cycles. The purpose of the much higher dose would be to insure both 100% inhibition viral replication and 100% inhibition of de novo synthesis of mediators of inflammation by lung resident macrophages and mast cells, the latter being able to reduce inflammation by up to 44% as shown in FIG. 3. Further anti-inflammatory activity would also be expected form inhibition of synthesis or viral proteins such as NS1, believed to be responsible for an enhanced proinflammatory cytokine response (especially TNF-alpha) in human macrophages, as previously disclosed.

Substituting the 50 ng/ml dose into the equations above translates into a 60,000 ng therapeutic dose, a 90,000 ng administered dose. Using the example of the Spiriva HandiHaler, the 90,000 ng administered dose would be packaged with excipient in the gelatin capsules, which would be inserted into the inhaler, pierced, and then inhaled.

Safety: From a worst case safety perspective, assuming 100% of the administered dose went directly to blood, this would be 45 times smaller than the NOEL dose (4,100,000÷90,000=45) and as shown in FIG. 6.

Summary of Novelty over Prior Art: The antiviral activity of DAS would inhibit synthesis of all 10 influenza viral proteins (unlike prior art drugs such as Tamiflu or Relenza which target some strains of a single protein). Because DAS targets the host ribosome (i.e. binds to the ribosomal peptidyl transferase site) rather than targeting the virus, the approach would be protein mutation insensitive, as the mutated proteins would still need to be synthesized on the host ribosomes. Inhibiting viral replication and spread limits the number of infected cells that eventually need to be killed by cytotoxic T-Cells, which in turn limits morbidity and mortality. The anti-inflammatory activity, of DAS (or any suitable trichothecene) would come in part from the inhibition of cytokine and leukotriene production by tissue resident mast cells and macrophages and in part from the inhibition of viral proteins such as NS1 which have been implicated with enhanced upregulation of cytokine production by macrophages. The dual anti-inflammatory effect would address the observed lethal aspect of the condition known as “the cytokine storm”.

Scope: The scope of addressable pulmonary indications is not intended be limited to H5N1 or Influenza. The compositions and methods presented above are applicable to any pulmonary virus that uses host ribosomes for translation of its RNA into viral proteins as DAS/trichothecenes bind to the peptidyl transferase site on ribosomes and prevent translation of RNA into proteins. The above is only a representative example for the purpose of fulfilling the reduction to practice requirement of instant application. The example would be just as applicable to any mutation of the of avian flu. Because the host ribosome is rendered non functional, the mutated RNA would also be prevented from being translated. Because the host ribosome is rendered non functional, any virus that requires use of host ribosomes for synthesis of its proteins would be inhibited. The compositions of present invention would cover a wide spectrum of virus as well as concurrent infections with different viruses. The dual action antiviral/anti-inflammatory would also have applicability in other situations. As an example, COPD (Chronic Obstructive Pulmonary Disease) is associated with frequent viral infections including influenza A, influenza B, rhinovirus, respiratory synctial virus (RSV), corona virus, and parainfluenza virus, all of which further impair an already impaired breathing situation. The dual action antiviral/anti-inflammatory activity of DAS (or any other suitable trichothecene) would also be ideal in this type of situation.

Related Patent Disclosure: Applicant has two patents related to use of trichothecenes in pulmonary indications. The two patents are patentably distinct as follows:

U.S. Pat. No. 7,015,244 ('244) teaches use of trichothecenes for an “Inhalable Chemical Debridement for COPD”. Patent '244 uses cytotoxic doses to depopulate the lungs of over proliferated tissue masses associated with COPD, in order to provide a lasting improvement in respiration. The above method does not use cytotoxic doses and teaches use as a dual action antiviral/anti-inflammatory.

U.S. Pat. No. 7,012,091 teaches use of trichothecenes as “Inhalable Inhibitors of Inflammation in the Respiratory Tract” for allergic reactions, asthma, and COPD related inflammation. Instant application, Example 3, teaches the use of trichothecenes for dual action antiviral and anti-inflammatory activity to reduce morbidity and mortality from viral lung infections, including pandemic type situations that involve mutated strains of influenza virus that may not even exist yet.

### OTHER EXAMPLES, APPLICATIONS AND EMBODIMENTS

It should be noted that the above are only a few representative examples of numerous possible embodiments of present invention are nothing should be construed as limiting the scope of present invention to only the representative examples presented above. A large array of pathogens may be addressed by compositions of present invention and various trichothecenes or combinations of trichothecenes may be used to address both the pathogen and related inflammation. Trichothecenes with longer inactivation times, such as T-2, may be substituted and the administration frequency reduced accordingly. Alternatively, macrocyclic trichothecenes may be used at IC50 (or any other suitable IC dose level) to further reduce the frequency of administrations. Combinations of trichothecenes may also be used. The present invention also envisions the possibility of mixing the trichothecene(s) with other compounds or substances, including combinations of trichothecenes, or substances that facilitate administration, facilitate or regulate the rate and/or depth of penetration and/or absorption of said trichothecene mycotoxins, increase efficacy of said mycotoxins, provide prophylactic activity against infection, or provide any other beneficial or synergistic function. The compounds collectively described above are termed herein “pharmaceutical compositions”. As an example, antibiotics may be included as part of the “pharmaceutical composition”. As another example, pharmaceutical composition may include any ingredients to facilitate or enhance penetration, distribution, or retention of therapeutics to a given target tissue mass. As another example, other agents that prevent inflammation or allergic response such as mast cell stabilizing agents cromolyn and nedocromil may also be included as part of the pharmaceutical composition. The examples provided in the application are only a few of the innumerable combinations and situations in which a multiple mechanism of action against underlying pathogen(s) and against inflammation may be used. Nothing in this application is intended to limit the potential uses of therapeutics of present invention for treatment of only the conditions disclosed in the examples.

Although the doses used in the above representative examples use only the PSR MOA of trichothecenes, the SHR (sulphydryl restriction) MOA of trichothecenes, which manifests at higher doses, may also be used for antibacterial activity. A representative example is provided in the next section below.

The concentrations and doses used in the examples should not be construed as “optimal”. As is customary under prior art, all dosages would be further refined and scrutinized by in vivo testing in suitable animal models or in Phase I and II clinical trials on humans as required by the FDA and the lowest concentrations suitable to achieve efficacy would likely be called “optimal”. Likewise, optimal doses may turn out to be much higher than those used in the examples. The doses presented in this application were done so to fulfill the reduction to practice requirement of this application and are not intended to imply an absolute standard or “optimal” dose but are merely some representative examples of efficacious, yet safe, embodiments of present invention.

### Summary of Novelty and Unobviousness:

The three major reasons for the unobviousness of present invention relative to prior art are related to 1) toxicity stigma, 2) unexplained untoward effects (i.e. PSR vs SHR effects), and 3) prior art's misconceptions about protein synthesis inhibition.

1) Toxicity stigma: Virtually every article on trichothecenes contains the words “toxin” or “mycotoxin” in the title, evoking a visceral negative perception of the compounds which has gone a long way to poison the well for pursuit of the therapeutic potential of these compounds. From perspective of present invention, prior art's definition of toxicity or “toxin” is arbitrary, inconsistent, and unrelated to anything, other than the potential for a compound to induce toxicity. From perspective of present invention toxicity is defined relative to a therapeutically beneficial dose. Drinking twice the recommended daily dose of water in one sitting or taking 10 times the recommended dose of acetaminophen (Tylenol) can kill a person, yet neither are categorized as toxins under prior art. In the representative examples of present invention, the therapeutic dermal doses of DAS were from hundreds to millions of times smaller than the NOEL dose, yet DAS is classified as a toxin.

This difference in perspective has been the major obstacle in development and commercialization of the enormous therapeutic potential of trichothecenes such as DAS. Of the potential private capital sources contacted the overwhelming reason given for hesitancy in funding was toxicity concerns. A denied biodefense research grant application for the Avian Flu indication included the same concern: “The lead compound, DAS, is a potent mycotoxin. Even if this compound is used at nanogram levels, there will be a serious safety issues.” Likewise, the parent application of instant application also elicited many of the same safety concerns, to which end applicant has filed this CIP which includes the safety margin as a multiple of human NOEL dose for each example (shown in FIG. 6), and an expanded section on the difference between PSR/SHR effects to correct half century old misconceptions about PSR (as prior art had attributed all the effects to PSR). The therapeutic dose levels that provide RNAi/PSR activity, without appreciable SHR activity, are also more clearly defined in the representative examples.

2) Unexplained Untoward Effects (now explained by applicant)—PSR v. SHR: Virtually all of the published articles and literature on trichothecenes disclose only the PSR MOA and view everything in that light. That left a lot of unexplained, low level, untoward effects, which contributed in part to the abandonment of further pursuit of the technology. Trichothecenes also bind to sulphydryl groups. The first publication applicant was able to find that disclosed this second, weaker SHR MOA was a RIVM report in 2002, (M. E. J Pronk et. al., “Toxicology and occurrence of nivalenol, fusarenon X, diacetoxyscirpenol, neosolaniol, and 3- and 15-acetyldeoxynivalenol: a review of six trichothecenes”, RIVM, PO Box 1, 3720 BA Bilthoven, Inspectorate for Health Protection and Veterinary Public Health, project #388802, RIVM report 388802024/2002). In light of this second, weaker MOA, all of the unexplained adverse effects become readily explainable as follows: Sulphydryl groups are primarily used for concentration gradient maintenance, transport of structural proteins, and are necessary for the integrity and polymerization of microtubules. More specifically, sulphydryl groups function in various transport processes such the Ca2+ pump ATPase, vacuolar ATPase, simple channels for water and urea, gated chloride dependent K+ channel flux, and a variety of carriers for glucose, nucleosides, choline, phosphate, anions, cations (Chou et. al., “Lysosomal sulphate transport is dependent upon sulphydryl groups”, Biochemical Journal, 1998 330 (713-717). Sulphydryl restriction is known to be particularly destructive to actively cycling cells.

On a systemic level, the effects of sulphydryl restriction would be expected to be similar to mercury poisoning, as mercury binds to sulphydryl groups. The hallmark of mercury poisoning in neural impairment. The term “mad as a hatter” originated from hat makers exposed to mercury vapors used to cure felt, as the workers appeared disturbed or mentally confused and had confused speech and blurred vision.

Mechanistically, neural and muscular impairment is consistent with the underlying SHR MOA as neurons are highly dependent on concentration gradient maintenance and its rapid restoration after firing. Ca2+ influx into neurons is used for neurotransmitter release to propagate signal transmission across the synapse and in muscles for signal transmission at the neuromuscular synapse as well as direct action on muscle contraction via its interaction with actin/myosin. Inhibiting Ca2+ pump ATPase, used to pump out the calcium influx, would impair the maintenance of concentration gradients and impair nerve and muscle function. These are exactly the types of symptoms that appeared in Phase I human trials as doses were escalated above the NOEL dose. Murphy et. al. (Murphy et. al., “Phase I Clinical Evaluation of Anguidine”, Cancer Treat Rep 62:1497-1502, 1978) observed symptoms including confusion, somnolence (sleepiness), hypotension and ataxia (inability to coordinate smooth muscle movement)—all consistent with the SHR MOA beginning to manifest at higher doses. At high concentrations of DAS, when Goodwin et. al. administered 6 mg/m2 by IV push, “CNS symptoms and hypotension were dose limiting” but when the same 6 mg/m2 dose was given by 8 hour infusion these symptoms disappeared leaving only the traditional PSR effects as “myelosuppression was unacceptable while other toxic manifestations were minimal” (Goodwin et. al., “Phase I evaluation of anguidine (diacetoxyscirpenol, NSC-141537)”, Cancer, 1978 July; 42(1); 23-6). Pushing a 6 mg/m2 dose into 5 liters of blood translates into a 2,000 ng/ml transient spike of DAS in the blood, which would then taper down to a 250 ng/ml concentration spike in the 40 liters of blood and extracellular fluid. The blood spike concentration is 100 times larger than the 20 ng/ml IC99 PSR concentration derived from ex-vivo studies and the extracellular concentration is more than 10 times greater that the PSR IC99 derived from ex-vivo studies. Accordingly, with 99% PSR by 20 ng/ml, the different set of symptoms that begin to manifest at 250 ng/ml to 2,000 ng/ml concentrations could be attributed to “something else”, and SHR exactly explains that “something else”.

On a sub cellular level, SHR's activity can be predicted from the known function of sulphydryl groups (e.g. maintenance of fluid balance and structural integrity of a cell). In dermal studies of Type A trichothecenes by topical application in animal models (hairless guinea pigs), progressively increased blistering is observed from around 20,000 ng/ml to 100,000 ng/ml and skin necrosis is observed. Blistering and necrosis are consistent with the SHR MOA, as cells loose fluid balance, structural integrity, and eventually rupture. While trichothecenes were known to induce apoptosis, e.g. (Victor I. Shifrin and Paul Anderson of Brigham and Women's Hospital, “Trichothecene Mycotoxins Trigger a Ribotoxic Stress Response that Activated c-Jun N-terminal Kinase and p38 Mitogen-activated Protein Kinase and Induces Apoptosis”, The Journal of Biological Chemistry, Vol. 274, No. 20, Issue of May 14, pp. 13985-13992, 1999) or (Nagase et. al. “Apoptosis induction by T-2 Toxin: Activation of Caspase-9, Caspase-3, adn DFF-40 CAC through cytosolic release of cytochrome c in HL-60 cells”, Bioscience, Biotechnology, and Biochemistry, Vol 65, No. 8, 2001) the SHR MOA explains the ability of trichothecenes to induce necrosis at high doses. Cell rupturing is a necrotic death.

Mechanistically, SHR's activity would also be expected to contribute to PSI's inhibition of DNA synthesis, and the modest inhibition of RNA synthesis, which had previously been attributed only as a secondary effect of protein synthesis inhibition (e.g. Thompson and Wannemacher). DNA replication requires massive synthesis of histone and other core proteins on which the DNA strands are tightly coiled. The mass of core proteins roughly equals the mass of DNA, and as such PSI inhibition would impair DNA synthesis. The G1-Phase arrest observed by Dosik et. al. is mechanistically consistent with PSI's effect on the cell cycle control system, as environment sensitive G1-Phase cyclins have short half lives (˜15 minutes for Cyclin D), with PSI resulting in rapid drops in cyclin levels, which in turn prevents hyperphosphorylation/removal of the Rb block, which in turn prevents activation of transcription factors (e.g. E2F-DP1), which in turn prevents a cell from entering the S-Phase of the cell cycle, which is the phase in which DNA is replicated. PSI would also directly inhibit synthesis of proteins required for entry into, or progression thorough, the S-Phase. Mechanistically, SHR impairs nucleoside transport. Nucleosides include cytidine, uridine, adenosine, guanosine, thymidine and inosine which are the molecular building blocks of DNA and RNA. Accordingly, SHR could be expected to contribute to impaired RNA and DNA synthesis.

In light of the dual PSR/SHR MOA, an additional potential advantage of using trichothecenes in antibacterial applications is revealed. Sulphydryl inactivation is antibacterial. Antibacterial irrigating solutions such as Sodium Hypochlorite used on infected root canals work by inactivating sulphydryl groups of essential bacterial enzymes (Estrela et. al., “Mechanism of Action of Sodium Hypochlorite”, Brazilian Dentistry Journal, 2002, 13(2): 113-117). Sulphydryl inactivating mercury compounds such a mercurochrome were widely used as topical antibacterial agents since their discovery in 1919, until the FDA removed them from its “generally considered safe” category in 1998 due to concerns about potential mercury poisoning.

As an example, an alternate embodiment of the “Individual Acne Dose” presented in Example 2, a much higher concentration may be substituted in order to provide antibacterial activity, in addition to the antiproliferative and anti-inflammatory activity provided by the PSR MOA. Doses above 250 ng/ml dose could be expected to start demonstrating weak SHR activity, and hence the start of weak antibacterial activity. This alternative acne example would provide all of the antiproliferative and anti-inflammatory benefits of PSR plus some level of SHR related antibacterial activity, while still maintaining a large margin of systemic safety.

The most important aspect of viewing the prior art studies in light of SHR MOA is that it explains a lot of the unexplained adverse systemic symptoms, which had incorrectly been assumed by prior art to be related to the PSR MOA.

3) Prior Art Misconceptions about Protein Synthesis Restriction/Inhibition

Perhaps the best way to explain prior art's misconception about PSR or PSI is to describe the visceral reaction to the term PSI versus RNAi. Propose using PSI in humans and the audience is mortified. Propose using RNAi in humans and the audience is elated. They are the same thing. DNA is transcribed into RNA which is then translated into proteins. While RNAi is received as the next “hot” technology, the endpoint of RNA inhibition is the same as PSI: DNA does not get expressed as proteins. Full spectrum RNA inhibition is exactly the same as full spectrum protein synthesis inhibition from the perspective of the end result. It does not matter whether the RNA is inhibited from being transcribed from the DNA or the RNA is inhibited from being translated into proteins at the ribosome, or the RNA is inhibited anywhere in-between these two points, the end result is the same: proteins are not synthesized. Because trichothecenes prevent translation of RNA into proteins at the ribosome, they pick up the additional advantage of being antiviral, which may or may not be the case for other RNAi or PSI approaches, depending on their MOAs.

Probing the visceral negative reaction to PSI evokes typical responses such as “the textbooks teach PSI as being highly toxic”. Indeed, horror stories can easily be traced back to compounds such as Ricin and macrocyclic trichothecenes, which share PSI as one of their MOAs. By association, apparently all compounds that employ a PSI MOA have been incorrectly lumped together by prior art as highly toxic substances to be avoided.

Once the non PSI effects such as SHR are taken out, the next crucial point prior art appears to have missed is that PSI is only toxic if it persists long enough (e.g. FIG. 1b for normal non cycling cells, FIG. 1a for cancer cells).

This in turn translates into the most crucial factor to know being the inactivation time (i.e. how long the PSI compound remains biologically active). This is the key determinant to whether or not a PSI compound will be toxic.

Despite the ease with which the intracellular inactivation time of trichothecenes can be ascertained (by using resumption of proteins synthesis as an indicator, as previously disclosed by applicant), and despite the enormous body of studies that have been published related to virtually every aspect of trichothecenes, applicant could not find any comprehensive study that computed the intracellular inactivation times of the various trichothecenes. Prior art has studied virtually every aspect of trichothecenes except the most important aspect: intracellular inactivation time. This is consistent with prior art's lack of understanding of PSI and its related toxicity.

Based on the disclosures made in this application, including FIGS. 1a and 1b, using only PSI dose levels (no SHR activity) the 100% PSI state can be maintained for up to 3 days in normal, non cycling cells, without being toxic. By around 5 days of sustained, 100% PSI, some level of cell mortality could be expected. As previously disclosed, macrocyclic trichothecenes (Type D) can have an intracellular inactivation time of 7+ days. Accordingly, a single administration of a sufficiently high dose of macrocyclic trichothecenes irreversibly results in a fairly large amount of cellular cytotoxicity where internalized. As previously disclosed, the Type A trichothecene DAS has an intracellular inactivation time of around half a day, which means a single administration of DAS (100% PSI, no SHR) will not cause cellular cytotoxicity. DAS can be administered twice daily for up to 3 days with no cytotoxicity.

Using DAS for antiviral and anti-inflammatory activity, without any cellular cytotoxicity, can mean the difference between life and death in conditions such as Avian flu. In attempting to ascertain the availability of DAS, applicant was informed the NIH had destroyed its supplies of DAS and DAS was put on the select agent list after 9/11. Destroying the last supplies of a drug that could very likely be the only thing that prevents mass mortality from an Avian flu pandemic makes a strong prima face case for prior art's abject lack of understanding of PSI's enormous therapeutic potential as well as prior art's lack of understanding of PSI and its toxicity.

1-4. (canceled)

5. A method of treating a viral respiratory tract infection that is caused by a virus that requires use of host ribosomes for its replication or spread, by targeted transient ribosomal inhibition consisting administration by inhalation a composition containing Diacetoxyscirpenol, to a patient in need thereof, in therapeutically effective amounts to transiently inhibit ribosomal function.

6-7. (canceled)
